(19) United States
(12) Patent Application Publication
(10) Pub. No.: US 2021/0244296 A1
(43) Pub. Date: Aug. 12, 2021

(54) SYSTEM AND METHOD FOR BLOOD PRESSURE MEASUREMENT, COMPUTER PROGRAM PRODUCT USING THE METHOD, AND COMPUTER-READABLE RECORDING MEDIUM THEREOF

(71) Applicant: Amengine Corporation, Zhubei City (TW)

(72) Inventors: Sheng-Chieh HUANG, Hsinchu (TW); Paul C.P. CHAO, Taipei City (TW); Yung Hua KAO, Hsinchu (TW); Pei-Yu CHIANG, Hsinchu (TW)

(21) Appl. No.: 17/242,906

(22) Filed: Apr. 28, 2021

Related U.S. Application Data
(63) Continuation of application No. 15/204,392, filed on Jul. 7, 2016.

(30) Foreign Application Priority Data
Sep. 16, 2015 (TW) 104130568

(51) Int. Cl. A61B 5/021; G16H 50/50; A61B 5/00; G16H 50/70; A61B 5/024

(52) U.S. Cl. CPC A61B 5/02108; A61B 5/02133; A61B 5/02125; G16H 50/50; A61B 5/6898; G16H 50/70; A61B 5/7264; A61B 5/024; A61B 5/7278

(57) ABSTRACT

The present invention provides a system and method for blood pressure measurement, a computer program product using the method, and a computer-readable recording medium thereof. The present invention uses a sensor to measure an electrophysiological signal and establishes a personalized cardiovascular model through a numerical method, and re-establishes the personalized cardiovascular model through an optimization algorithm. Thus, a human physiological parameter generated from the re-established personal cardiovascular model matches the electrophysiological signal. Therefore, the present invention can provide accurate measurement results with the advantage of a small size, and can be applied to telemedicine field.

### RELATED APPLICATION DATA

This application is a continuation of U.S. application Ser. No. 15/204,392 filed Jul. 7, 2016, which claims the right of priority of TW Application No. 104130568, filed on Sep. 16, 2015, the contents of which are hereby incorporated by reference in their entireties.

### BACKGROUND OF THE INVENTION

### Field of the Invention

The present invention relates to blood pressure measurement, and, more particularly, to a system and method for blood pressure measurement that establishes a personalized cardiovascular model using a finite element method, a computer program product using the same, and a computer-readable recording medium thereof.

### Description of Related Art

Due to changes in modern lifestyle and refined diet, there has been a substantial increase in various chronic diseases, such as hypertension, diabetes, high cholesterol or cardiovascular diseases and the like. In pursuit of health, there are more and more passive electronic physiological measuring instruments emerging on the market that allow people to monitor their health in their own homes, enabling convenient and rapid physiological measurements.

In terms of measuring the blood pressure, mercury sphygmomanometers and oscillometric blood pressure monitors, for example, are commercially available. Although mercury sphygmomanometers provide accurate blood pressure measurement results, it requires professional personnel or trained person to carry out the operation, otherwise it is difficult to obtain satisfactory measurements. As a result, users cannot monitor their own health at any time. Oscillometric blood pressure monitors are more convenient to carry and operate than mercury sphygmomanometers, but as they employ the principle of oscillation for detection, an oscillometric blood pressure monitor has to be bound to the arm via a cuff, and the tightness of the cuff will affect the magnitude of the blood pressure measured, leading to low accuracy in the monitoring results.

Therefore, there is a need for a blood pressure measuring technique that addresses the aforementioned issues in the prior art.

### SUMMARY OF THE INVENTION

In view of the aforementioned shortcomings of the prior art, a main objective of present invention is to provide a method for blood pressure measurement, which may include: measuring an electrophysiological signal and obtaining basic personal information; establishing a personalized cardiovascular model based on the electrophysiological signal and the basic personal information through a numerical method, wherein the personalized cardiovascular model includes default model parameters and is used to create a human physiological parameter; and calibrating the default model parameters through an optimization algorithm to re-establish the personalized cardiovascular model, such that the human physiological parameter created by the re-established personalized cardiovascular model matches the measured electrophysiological signal.

Another objective of present invention is to provide a system for blood pressure measurement, which may include: a measuring unit for measuring an electrophysiological signal and including a user interface allowing basic personal information to be input thereon; and a computing unit connected with the measuring unit via a network for receiving the electrophysiological signal and the basic personal information. The computing unit may include: a modeling module for establishing a personalized cardiovascular model based on the electrophysiological signal and the basic personal information through a numerical method, wherein the personalized cardiovascular model includes default model parameters and is used to create a human physiological parameter; and an optimization module for calibrating the default model parameters through an optimization algorithm to re-establish the personalized cardiovascular model, such that the human physiological parameter created by the re-established personalized cardiovascular model matches the measured electrophysiological signal.

Yet another objective of present invention is to provide a computer program product for executing the method for blood pressure measurement described previously after being loaded into a machine.

Still another objective of present invention is to provide a computer-readable recording media, within which a computer program is stored. The computer program, after being loaded into a machine, is configured to execute the method for blood pressure measurement described previously.

In the system and method for blood pressure measurement, the computer program product that uses the method, and the computer readable recording medium thereof in accordance with the present invention, the blood pressure measuring sensor used is in the form of a vibrating pulse sensor or an optical sensor. The present invention thus eliminates the inconvenience associated with traditional cuff-type blood pressure measuring devices. The present invention also has a smaller volume which makes it easier to carry. In addition, the present invention allows the establishment of the personalized cardiovascular model in the cloud server and the calibration of the personalized cardiovascular model through the optimization algorithm. As a result, the human physiological parameter created by the personalized cardiovascular model can be more accurate. Furthermore, the personalized cardiovascular model can be used in the telemedicine field, facilitating long-range medical care services and early warning systems.

### BRIEF DESCRIPTION OF THE DRAWINGS

The present invention can be more fully understood by reading the following detailed description of the preferred embodiments, with reference made to the accompanying drawings, wherein:

FIG. 1 is a flowchart illustrating the steps of a method for blood pressure measurement in accordance with the present invention; and

FIG. 2 is a block diagram depicting a system for blood pressure measurement in accordance with the present invention.

### DETAILED DESCRIPTION OF THE EMBODIMENTS

The present invention is described by the following specific embodiments. Those with ordinary skills in the arts can readily understand other advantages and functions of the present invention after reading the disclosure of this specification. The present disclosure may also be practiced or applied with other different implementations.

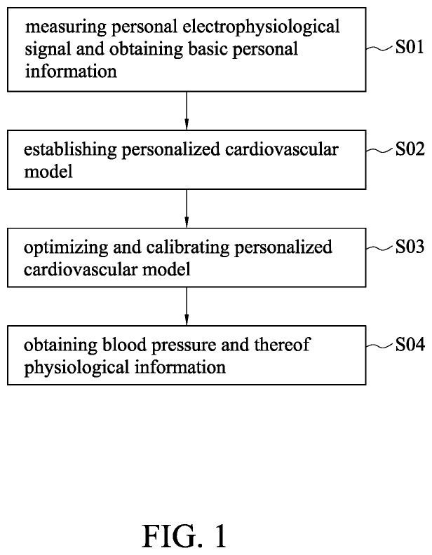

Referring to FIG. 1, a method for blood pressure measurement in accordance with the present invention includes: measuring an electrophysiological signal and obtaining basic personal information of an individual (step S01); establishing a personalized cardiovascular model (step S02); optimizing and calibrating the personalized cardiovascular model (step S03); and obtaining blood pressure and physiological information thereof (step S04).

In step S01, a personal electrophysiological signal is measured and basic personal information is obtained. More specifically, a pulse signal is measured by a sensor as the personal electrophysiological signal. The sensor may be a vibrating pulse sensor or an optical sensor.

In use, a vibrating pulse sensor is first attached to the wrist, and a pulse signal is acquired via a vibrating sensing unit in the sensor. An optical sensor can be a camera in a smartphone. During measurement, the camera obtains an optical signal that is not visible to the human eyes, and the optical signal is then converted into a personal pulse signal through an image processing algorithm.

In one embodiment, the vibrating pulse sensor can be a smartwatch. A pulse signal, after obtained, is wirelessly transmitted to a cloud server (e.g., infrared, Bluetooth, WiFi etc., but the present invention is not limited as such). Alternatively, the pulse signal is transmitted to a nearby smartphone first, and then transmitted to a cloud server via the smartphone. In one embodiment, the pulse signal may be transmitted to a nearby computer or a WiFi sharing device first, and then transmitted to a cloud server. The optical sensor may transmit the pulse signal to a cloud server directly from the smartphone.

Before or after the personal electrophysiological signal is measured, basic personal information is obtained via a user interface. In an embodiment, the basic personal information may include, but not limited to information such as age, height, weight, gender, body fat ratio etc., and/or personal information contained in the cloud electronic medical records. The user interface is a data input interface provided by an app in the smartwatch or the smartphone.

In step S02, after obtaining the personal electrophysiological signal and basic personal information, a personalized cardiovascular model is built by a numerical method. In an embodiment, the personalized cardiovascular model has default model parameters, and creates a human physiological parameter. In establishing the personalized cardiovascular model, based on the basic personal information of the individual, parameters such as vascular wall thickness, vessel diameter, vessel density, blood vessel elasticity, blood concentration, blood density, blood viscosity coefficient, blood flow, blood flow field pressure, muscle elasticity and density, elasticity and density of the bones, skin elasticity, or elasticity and density of all the tissues from the wrist continuously extended to the heart are set as the default model parameters of the personalized cardiovascular model. These default model parameters will have different default values depending on the parameters such as age, height, weight and gender. Based on these default model parameters, a personalized cardiovascular model can be built by a numerical method, e.g., the finite element method. In one embodiment, the personalized cardiovascular model is an arterial finite element model, and is built from the measuring point (i.e., the wrist).

In one embodiment, the personalized cardiovascular model may be further based on a new Blood Pressures Transport Theory (BPTT) modeled from the wrist continuously extending to the heart, and its model parameters are calibrated according to the size and direction of the pulse wave vector in the measured pulse signal. This ultimately becomes the personalized cardiovascular finite element model. The personalized cardiovascular model can be formed into a personalized radial artery cardiovascular finite element model.

After the personalized cardiovascular model is acquired, the human physiological parameter of blood pressure (BP) can be obtained from the following formula:

BP=C(½ρd²/PTT²+ρgh),

wherein C is the cardiovascular constant, ρ the blood density, d the blood vessel distance between the heart and the measuring point, PTT the average velocity of blood from the heart to the measuring point, g the gravitational constant, and h the height difference between the heart and measuring point.

In step S03, as the personalized cardiovascular model is initially built based on the default values of the model parameters that correspond to the basic personal information (such as age, height, weight, gender, body fat ratio, or data contained in the cloud electronic medical records etc.), it may not accurately reflect the individual status using the same default values, since persons have different cardiovascular status in reality, the default model parameters are calibrated using an optimization algorithm so the personalized cardiovascular model can better match the personal electrophysiological signal measured.

In one embodiment, the optimization algorithm is a genetic algorithm, a neural network algorithm, an intelligent algorithm, or any novel algorithm that automatically optimizes and calibrates the default values of the model parameters using a large number of samples.

In another embodiment, physiological information, such as the systolic blood pressure, the diastolic blood pressure, or the heart rate obtained from, for example, health checks or other blood pressure measuring devices can be used as the basis for calibrating the electrophysiological signal, and a more accurate formula for calculating blood pressure can be formed through the self-calibrating optimization algorithm as described before.

In step S04, based on the adjusted model parameters, the personalized cardiovascular model is re-established, such that the human physiological parameter created from the re-established personalized cardiovascular model matches the electrophysiological signal of the individual, and measurement results of the physiological information (e.g., the systolic blood pressure, the diastolic blood pressure or the heart rate) can be obtained.

The present invention also provides a computer program product for executing the method for blood pressure measurement previously described after being loaded into a machine (such as a computer or a smart phone). The present invention further provides a computer-readable recording media, such as a CD, a DVD, a flash drive, a hard drive, etc., within which a computer program is stored, and the computer program, after being loaded into a machine (such as a computer or a smart phone), is used for executing the method for blood pressure measurement previously described.

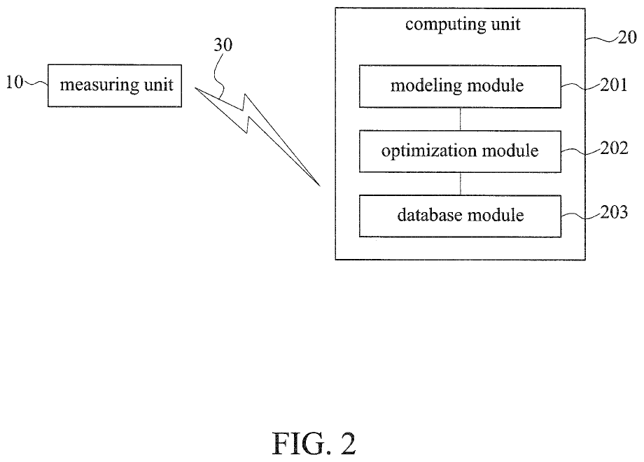

In FIG. 2, a blood pressure measurement system in accordance with the present invention includes a measuring unit 10 and a computing unit 20 is shown. The measuring unit 10 and the computing unit 20 are connected through a network 30. The measuring unit 10 is, for example, a vibrating pulse sensor of a smartwatch, or an optical sensor of a camera of a smartphone.

The measuring unit 10 is used for measuring a personal electrophysiological signal, such as a pulse signal, the systolic blood pressure, the diastolic blood pressure, or the heart rate. The computing unit 20 is a personal computer (PC) or a cloud server. In one embodiment, the measuring unit 10 has a user interface provided by a software program to allow basic personal information to be input thereon. In an embodiment, the basic personal information includes parameters such as age, height, weight, gender, body fat ratio, and/or data contained in cloud electronic medical records.

The computing unit 20 receives the electrophysiological signals and the basic personal information from the measuring unit 10 via the network 30. The computing unit 20 includes a modeling module 201, an optimization module 202 and a database module 203. The modeling module 201 and the optimization module 202 are software programs written in C, C++, C #, JAVA, Python, or other programming languages supporting network connection function, and the database module 203 is a HIS, NIS, HL7, or a general database (such as MySQL, SQL Server, Oracle, or Microsoft Access). The format in which the measuring unit 10 transmits the electrophysiological signal and the basic personal information to the computing unit 20 may be, but not limited to JSON, XML, YAML or other customized formats.

In one embodiment, the network 30 is, but not limited to a wireless LAN, an infrared or Bluetooth wireless network, or a wired network.

The modeling module 201 establishes a personalized cardiovascular model through a numerical method based on the electrophysiological signal and the basic personal information. In an embodiment, the personalized cardiovascular model has default model parameters, and creates a human physiological parameter. In one embodiment, the numerical method is a finite element method. The details about how to establish a personalized cardiovascular model and obtain a human physiological parameter therefrom have been described above, further description hereby omitted.

The optimization module 202 calibrates the default model parameters through an optimization algorithm and re-establishes the personalized cardiovascular model. In an embodiment, the optimization algorithm is a genetic algorithm, a neural network algorithm, an intelligent algorithm, or any novel algorithm that automatically optimizes and calibrates the default values of the model parameters using a large number of samples, such that the human physiological parameter created by the re-established personalized cardiovascular model matches the measured personal electrophysiological signal.

In one embodiment, the human physiological parameter is the systolic blood pressure, the diastolic blood pressure, or the heart rate. The default model parameters includes, bur is not limited to vascular wall thickness, vessel diameter, vessel density, blood vessel elasticity, blood concentration, blood density, blood viscosity coefficient, blood flow, blood flow field pressure, muscle elasticity and density, elasticity and density of the bones, skin elasticity or elasticity and density of all the tissues from the wrist continuously extended to the heart.

The database module 203 is used for storing the measured electrophysiological signal, the inputted basic personal information, the established personalized cardiovascular model, and the optimized and re-established personalized cardiovascular model.

The system and method for blood pressure measurement, the computer program product that uses the method, and the computer readable recording medium thereof in accordance with the present invention eliminates the use of the cuff by measuring the personal electrophysiological signal with a measuring unit in the form of a vibrating pulse sensor or an optical sensor. The measuring unit further provides a user interface that facilitates user operations, input of the basic personal information and display of measurement results. The present invention thus eliminates the inconvenience associated with traditional cuff-type blood pressure measuring devices. The present invention also has a smaller volume which makes it easier to carry. The measured personal electrophysiological signal and the inputted basic personal information can be transmitted to a cloud server for integration and operation in order to establish a personalized cardiovascular model, and then the human physiological parameter (i.e., the blood pressure) can be provided to the user based on the modeling results.

In addition, the system for blood pressure measurement in accordance with the present invention can be further combined with the database of a medical center to establish long-range medical care services and early warning systems, and the personalized cardiovascular model is continuously updated by the optimization algorithm to get the latest accurate blood pressure, thereby achieving health management and care anytime and anywhere based on the blood pressure.

The above embodiments are only used to illustrate the principles of the present invention, and should not be construed as to limit the present invention in any way. The above embodiments can be modified by those with ordinary skill in the art without departing from the scope of the present invention as defined in the following appended claims.

What is claimed is:

1. A method for measuring a blood pressure of a human who has a heart and a wrist, comprising:
providing an initial personal electrophysiological signal by measuring a pulse signal from a sensor device which is worn on the wrist;
providing a basic personal information through an electronic device;
storing the initial personal electrophysiological signal and the basic personal information in a cloud server;
establishing a personalized cardiovascular model built from the wrist continuously extending to the heart and implemented by the cloud server, wherein the personalized cardiovascular model is constituted as a finite element model, wherein the finite element model is set by a plurality of model parameters to distill a physiological information of the human, wherein the plurality of model parameters is built based on the initial personal electrophysiological signal and the basic personal information;
calibrating and optimizing the personalized cardiovascular model through an optimization algorithm in the cloud server to obtain an adjusted plurality of model parameters;
establishing a re-established cardiovascular model set by the adjusted plurality of model parameter and a latest personal electrophysiological signal;
measuring the latest personal electrophysiological signal from the sensor device; and
obtaining the blood pressure from the physiological information of the human through the re-established cardiovascular model.

2. The method of claim 1, wherein the sensor device is a vibrating pulse sensor which is configured to attach to the wrist.

3. The method of claim 2, wherein the vibrating pulse sensor is a smart watch.

4. The method of claim 1, wherein the sensor device is an optical sensor configured to obtain an optical signal from the wrist and convert the optical signal into the pulse signal.

5. The method of claim 1, wherein the basic personal information includes age, height, weight, gender, body fat ratio.

6. The method of claim 1, wherein the plurality of model parameters includes a vascular wall thickness, a vessel diameter, a vessel density, a blood vessel elasticity, a blood concentration, a blood viscosity coefficient, a blood flow, a blood flow field pressure, a muscle elasticity and density, a elasticity and density of bones, a skin elasticity, or a elasticity and density of all tissues from the wrist continuously extending to the heart.

7. The method of claim 1, wherein the physiological information of the human includes a systolic blood pressure and a diastolic blood pressure.

8. The method of claim 1, wherein the optimization algorithm comprises a genetic algorithm, a neural network algorithm, or an intelligent algorithm.

9. The method of claim 1, further comprising measuring the physiological information of the human through a measuring device different from the sensor device to calibrate the personalized cardiovascular model in the calibrating and optimizing step.

10. The method of claim 9, wherein the measuring device can be a blood pressure measuring device.

11. The method of claim 1, wherein the personalized cardiovascular model is built by a finite element method.

12. The method of claim 1, further comprising continuously measuring the latest personal electrophysiological signal for continuously calibrating and optimizing the personalized cardiovascular model.

13. A method for measuring a blood pressure of a human who has a heart and a wrist, comprising:
providing a personal electrophysiological signal retrieved from a sensor device which is exclusively placed on the wrist, and a basic personal information through an electronic device;
storing the personal electrophysiological signal and the basic personal information in a cloud server;
establishing a personalized cardiovascular model continuously built from the wrist to the heart;
optimizing the personalized cardiovascular model by using the personal electrophysiological signal and the basic personal information stored in the cloud sever and obtain an optimized personalized cardiovascular model; and
obtaining the blood pressure by applying another personal electrophysiological signal from the sensor device to the optimized personalized cardiovascular model.

14. The method of claim 13, wherein the sensor device is a vibrating pulse sensor which is configured to attach to the wrist.

15. The method of claim 14, wherein the vibrating pulse sensor is a smart watch.

16. The method of claim 13, wherein the sensor device is an optical sensor configured to obtain an optical signal from the wrist and convert the optical signal into the pulse signal.

17. The method of claim 13, wherein the basic personal information includes age, height, weight, gender, or body fat ratio.

18. The method of claim 13, wherein the personalized cardiovascular model is built by a finite element method.

19. The method of claim 13, wherein the personalized cardiovascular model is optimized by using a plurality of personal electrophysiological signals.
